(19) United States
(12) Patent Application Publication
(10) Pub. No.: US 2026/0227896 A1
(43) Pub. Date: Aug. 6, 2026

(54) PHYSIOLOGICAL MONITOR TOUCHSCREEN INTERFACE

(71) Applicant: MASIMO CORPORATION, Irvine, CA (US)

(72) Inventors: Ammar Al-Ali, San Juan Capistrano, CA (US); Bilal Muhsin, San Clemente, CA (US); Keith Ward Indorf, Riverside, CA (US)

(21) Appl. No.: 19/435,590

(22) Filed: Dec. 29, 2025

Related U.S. Application Data
(63) Continuation of application No. 17/449,069, filed on Sep. 27, 2021, now Pat. No. 12,541,293.
Application No. 17/449,069 is a continuation of application No. 16/677,483, filed on Nov. 7, 2019, now Pat. No. 11,132,117.
Application No. 16/677,483 is a continuation of application No. 14/949,701, filed on Nov. 23, 2015, now Pat. No. 10,503,379.
Application No. 14/949,701 is a continuation of application No. 13/850,000, filed on Mar. 25, 2013, now Pat. No. 9,195,385.
(60) Provisional application No. 61/615,307, filed on Mar. 25, 2012.
(60) Provisional application No. 61/615,316, filed on Mar. 25, 2012.
(60) Provisional application No. 61/615,876, filed on Mar. 26, 2012.

(51) Int. Cl. G06F 3/04847; G06F 3/04817; G06F 3/0482; G06F 3/04845; G06F 3/0485; G06F 3/0486; G06F 3/0488; G16H 40/63

(52) U.S. Cl. CPC G06F 3/04847; G06F 3/04817; G06F 3/0482; G06F 3/04845; G06F 3/0485; G06F 3/0486; G06F 3/0488; G16H 40/63

(57) ABSTRACT

A physiological monitor touchscreen interface presents interface constructs on a touchscreen display that are particularly adapted to finger gestures so to change at least one of a physiological monitor operating characteristic and a physiological touchscreen display characteristic.

### CROSS-REFERENCE TO RELATED APPLICATIONS

The present application is a continuation of U.S. patent application Ser. No. 17/449,069, filed Sep. 27, 2021, titled Physiological Monitor Touchscreen Interface, which is a continuation of U.S. patent application Ser. No. 16/677,483, filed Nov. 7, 2019, now U.S. Pat. No. 11,132,117, titled Physiological Monitor Touchscreen Interface, which is a continuation of U.S. patent application Ser. No. 14/949,701, filed Nov. 23, 2015, now U.S. Pat. No. 10,503,379, titled Physiological Monitor Touchscreen Interface, which is a continuation of U.S. patent application Ser. No. 13/850,000, filed Mar. 25, 2013, now U.S. Pat. No. 9,195,385, titled Physiological Monitor Touchscreen Interface, which claims priority benefit under 35 U.S.C. § 119(e) to U.S. Provisional Patent Application Ser. No. 61/615,307, filed Mar. 25, 2012, titled Physiological Monitor User Controls; U.S. Provisional Patent Application Ser. No. 61/615,316, filed Mar. 25, 2012, titled Physiological Monitor User Interface; and U.S. Provisional Patent Application Ser. No. 61/615,876, filed Mar. 26, 2012, titled Physiological Monitor Touchscreen; all of the above referenced applications are hereby incorporated in their entireties by reference herein.

### BACKGROUND

Pulse oximetry is a widely accepted noninvasive procedure for measuring the oxygen saturation level of arterial blood, an indicator of a person's oxygen supply. A typical pulse oximetry system utilizes an optical sensor attached to a fingertip to measure the relative volume of oxygenated hemoglobin in pulsatile arterial blood flowing within the fingertip. Oxygen saturation (SpO₂), pulse rate and a plethysmograph waveform, which is a visualization of pulsatile blood flow over time, are displayed on a monitor accordingly.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated in their entirety by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entirety by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are described in at least U.S. Pat. No. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc., Irvine, CA (Cercacor) and all incorporated in their entirety by reference herein. Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Such advanced pulse oximeters, low noise sensors and advanced blood parameter systems have gained rapid acceptance in a wide variety of medical applications, including surgical wards, intensive care and neonatal units, general wards, home care, physical training, and virtually all types of monitoring scenarios.

### SUMMARY

A physiological monitor touchscreen interface presents interface constructs on a touchscreen display that are particularly adapted to finger gestures so to change at least one of a physiological monitor operating characteristic and a physiological touchscreen display characteristic. The physiological monitor touchscreen interface has a first interface construct operable to select a menu item from a touchscreen display and a second interface construct operable to define values for the selected menu item.

In various embodiments, the first interface construct has a first scroller that presents a rotating set of the menu items in a touchscreen display area and a second scroller that presents a rotating set of thumbnails in a display well. The thumbnails reference the menu items and the thumbnails rotate with the menu items. The first scroller presents a rotating set of second level menu items upon selection of the menu item. The second interface construct is a slider for selecting limits for one of the second level menu items. A spinner is used in conjunction with the slider for making a first gross limit selection with the slider followed by a finer limit selection with the spinner. A parameter area displays parameter values in a full presentation format and a parameter well area displays parameter values in a abbreviated presentation format. The full presentation format is a larger font that the abbreviated presentation format. A dynamic space allocation for the parameters values is presented in the parameter area such that the more parameters there are in the parameter area and, accordingly, the fewer parameters there are in the parameter well area, then the larger the display font for the parameters in the parameter area.

### BRIEF DESCRIPTION OF THE DRAWINGS

FIG. 1 is a hierarchical chart of a physiological monitor touchscreen interface including interface constructs and finger controls;

FIGS. 2A-7D are illustrations of various touchscreen interface constructs for controlling a physiological monitor;

FIGS. 2A-D are illustrations of a scroller;

FIGS. 3A-C are illustrations of a physiological monitor main menu and sub-menus implemented with a scroller;

FIGS. 4A-C are illustrations of a spinner;

FIGS. 5A-B are illustrations of a slider;

FIG. 6 is an illustration of a slider-spinner; and

FIGS. 7A-D are illustrations of a parameter monitor touchscreen providing dynamic allocation of the parameter display area utilizing a parameter well.

### DETAILED DESCRIPTION

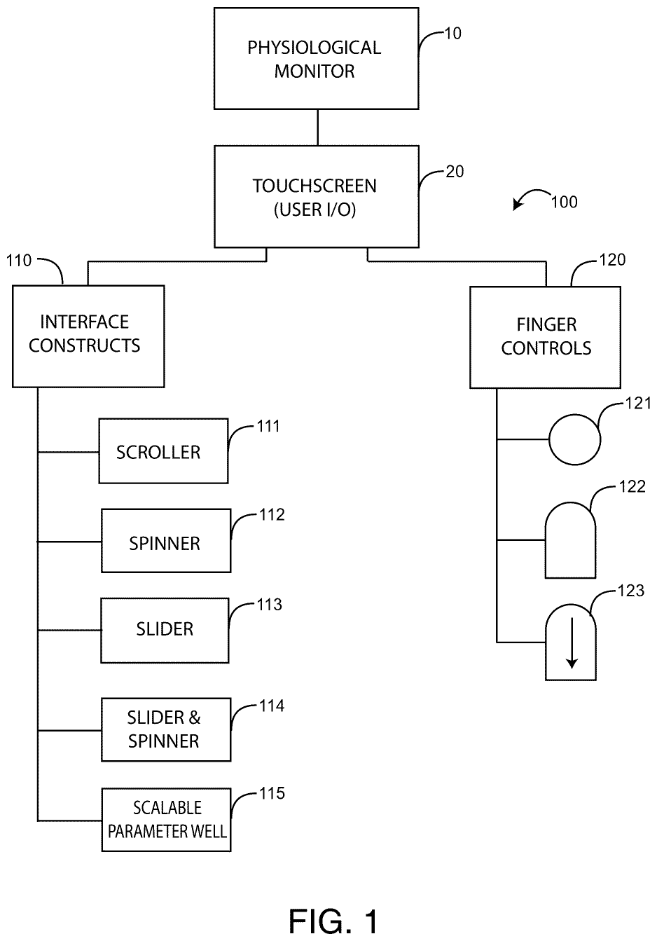

FIG. 1 illustrates a touchscreen interface 100 for a physiological monitor 10 and, in particular, for a touchscreen display 20 integral to the monitor 10. In general, the touch screen interface 100 provides an intuitive, gesture-oriented control for the physiological monitor 10. In particular, the touchscreen display 20 presents a user with interface constructs 110 responsive to finger controls 120 so as to change displays and settings, such as monitor operating characteristics, display contents and display formats using a finger touch, a finger touch and move, or a fingertip flick.

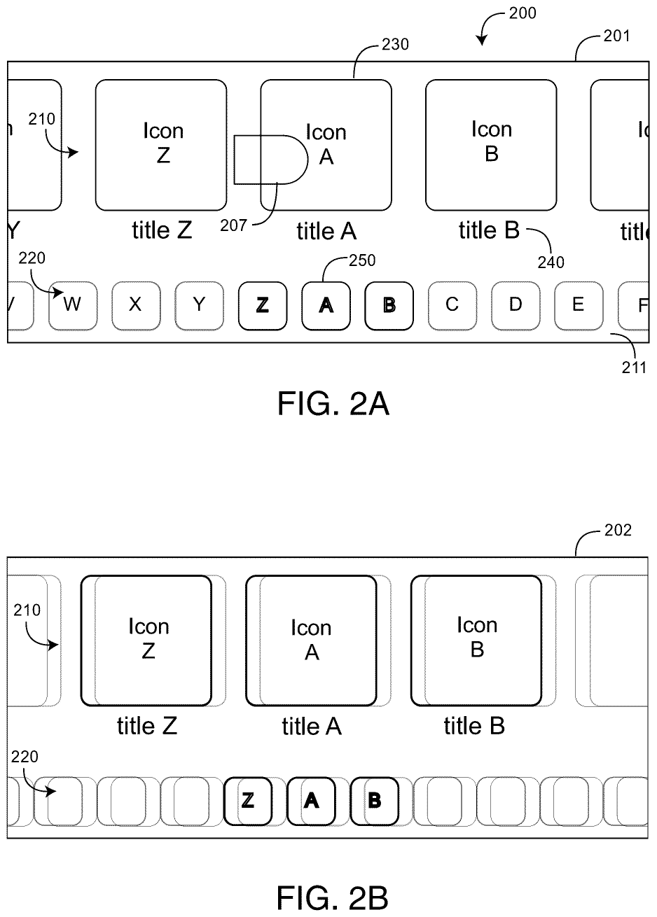

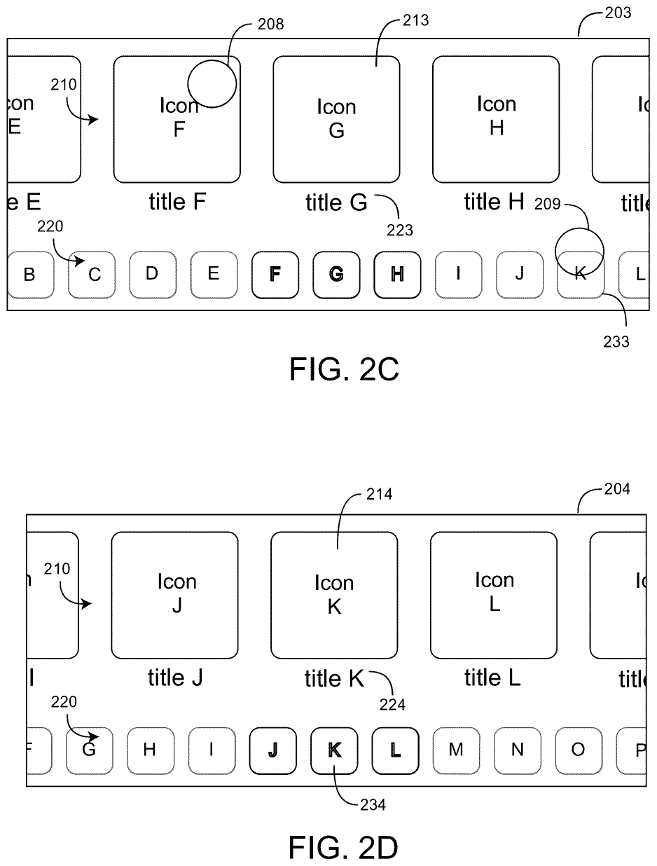

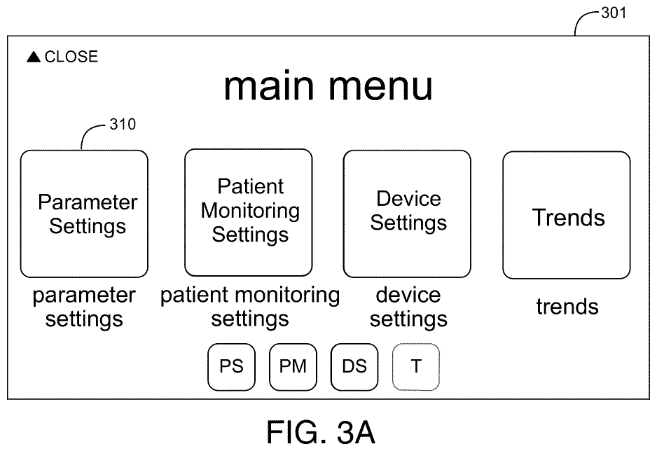

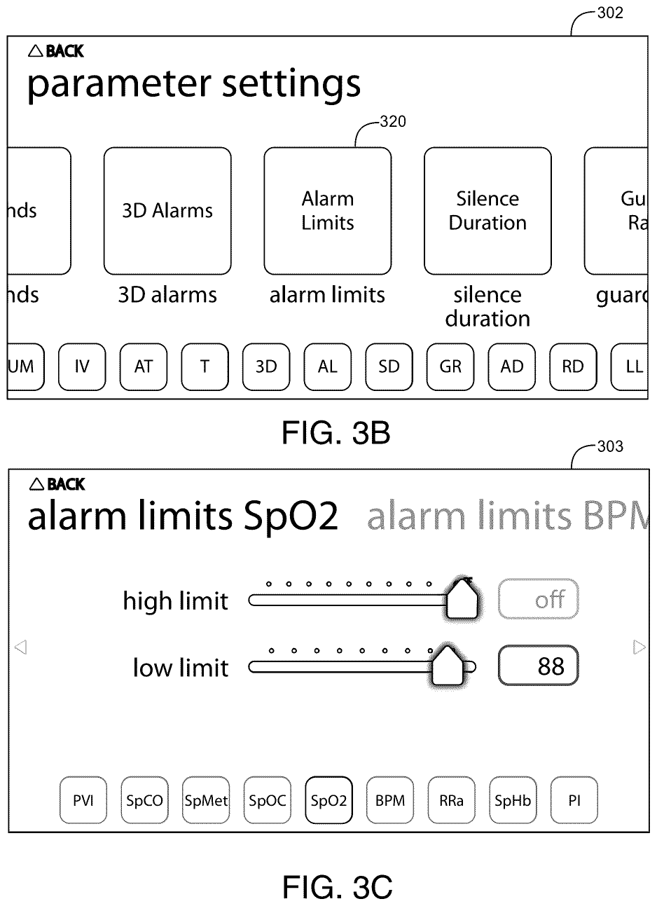

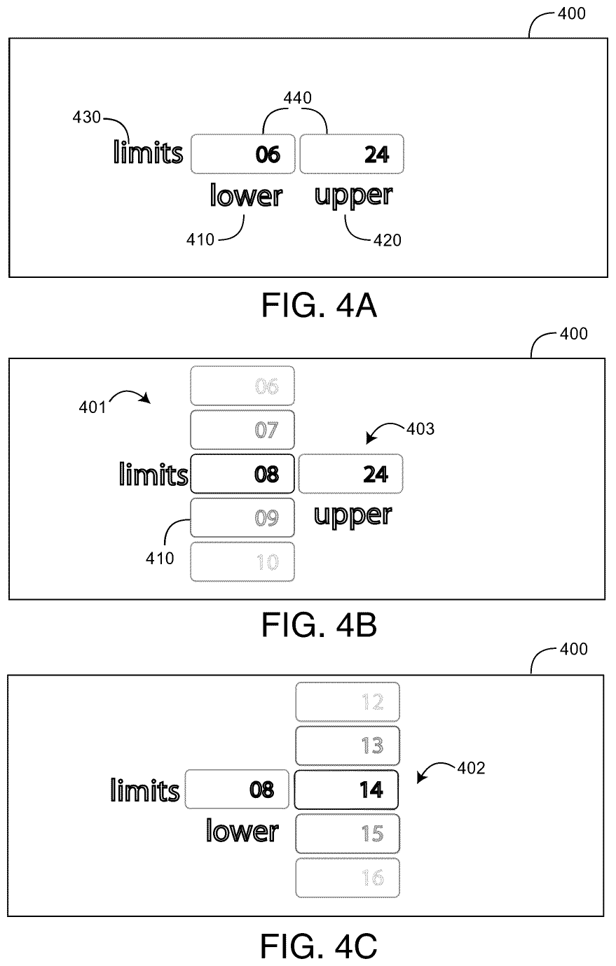

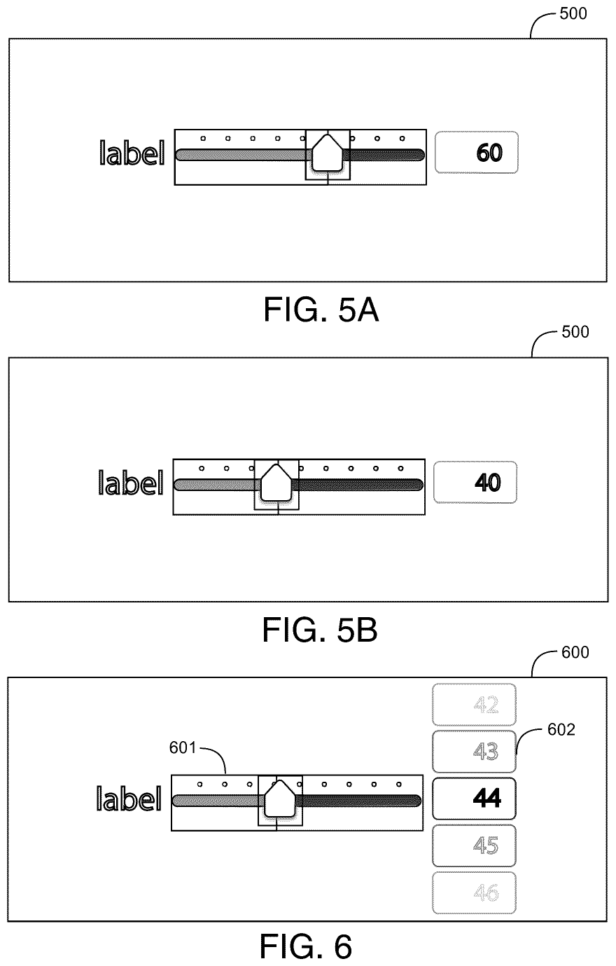

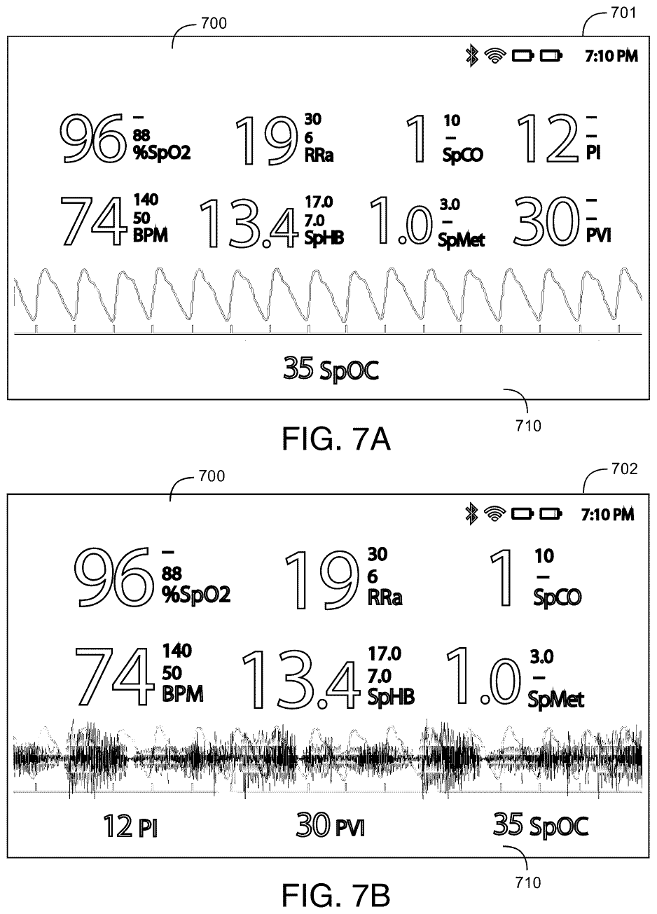

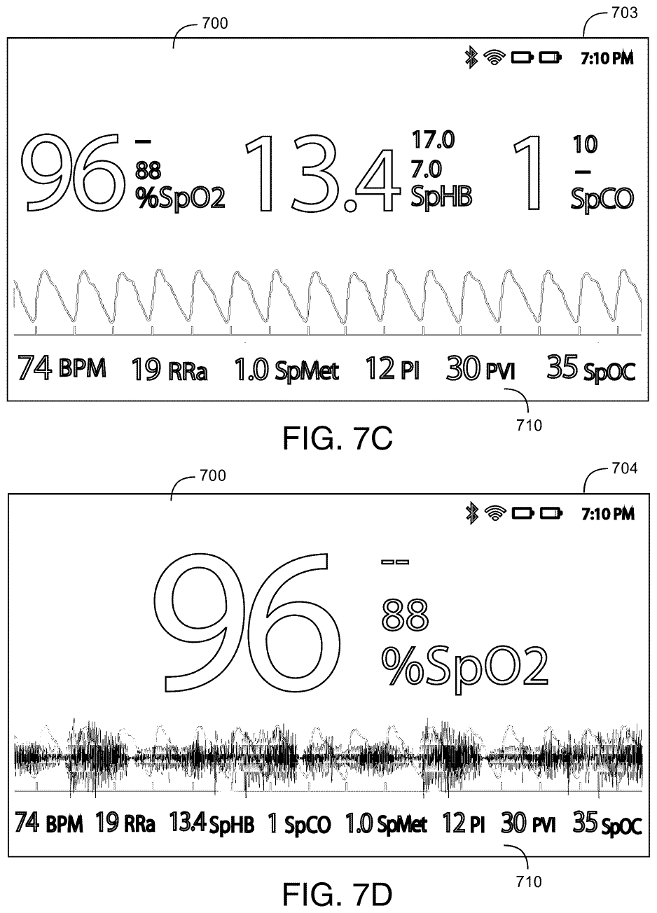

As shown in FIG. 1, interface constructs 110 include a scroller 111, a spinner 112, a slider 113, a slider-spinner 114 and a scalable parameter well 115. A scroller 111 is described below with respect to FIGS. 2-3. A spinner 112 is described below with respect to FIGS. 4A-C. A slider 213 is described below with respect to FIGS. 2-3. A slider-spinner 214 is described below with respect to FIGS. 5A-B. A scalable parameter well 216 is described below with respect to FIGS. 7A-D.

Also shown in FIG. 1, finger controls 120 include a touch 121, a touch and move 121 and a flick 121. A touch 121 is finger contact with an active display area. A touch and move 121 is finger contact in conjunction with finger movement in a particular direction. A flick 121 is finger contact in conjunction with a quick finger movement in a particular direction.

FIGS. 2-7 illustrate various touchscreen interface constructs 110 (FIG. 1) for controlling a physiological monitor 10 (FIG. 1), as described above. FIGS. 2A-D illustrate a scroller 200 construct configured for a touchscreen display. The scroller 200 is organized as a menu 210 disposed on a virtual, horizontally-rotatable loop. Only a viewable section 201-204 of the menu 210 is visible on the display at any given time. The scroller 200 is responsive to finger controls so as to bring into view any menu section, as described below.

Also shown in FIGS. 2A-D, a scroller 200 embodiment has thumbnails 250 disposed on a second, virtual, horizontally-rotatable loop located in a display well 211. The menu 210 has menu icons 230 and corresponding menu titles 240. The thumbnails 250 have a one-to-one correspondence to the menu icons 230, as indicated by thumbnail icons corresponding to the menu icons or thumbnail initials corresponding to the menu titles 240.

Further shown in FIGS. 2A-D, the scroller 200 advantageously allows for an unrestricted number of menu items. A user can rotate the scroll left or right using touch and move 122 (FIG. 1). A user can scroll left or right with velocity using flick 123 (FIG. 1). Further, a user can navigate to a menu item using touch 121 (FIG. 1) on menu item icon or title. In addition, a user can quick scroll to menu item using touch 121 (FIG. 1) on a thumbnail in the display well 211.

As shown in FIGS. 2A-D, when the user applies touch and move 207 (FIG. 2A) to the menu icons the user can freely and smoothly slide the menu 201 (FIG. 2A) to the left 202 (FIG. 2B) or the right. On release the menu icons snap and lock 203 (FIG. 2C) to their closest grid location employing an ease-in animation so the transition is smooth and natural and not abrupt. Then, on touch 208 (FIG. 2C) the user can navigate to any visible menu option 210. The navigate executes on release.

Further shown in FIGS. 2C-D, when the user wants to jump to a menu item not on the screen they can use a quick scroll. The user applies touch 209 (FIG. 2C) on a particular thumbnail indicator (K) 233 and the icon menus scroll into position giving center focus to the menu item (Icon K) 214 FIG. 2D represented by the touched thumbnail indicator 233. As shown in FIG. 2D, once the icon menu scroll animation is complete, the thumbnail indicators rapidly slide to their new orientation.

When the user applies flick (not shown) to the menu icons 210, the menu icons move with velocity along the horizontal vector the gesture implied and the icon menus slide into place. In particular, when the menu icons momentum decreases and they begin to come to a stop, the menu icons will snap to their closest grid location as described above.

FIGS. 3A-C illustrate a physiological monitor main menu 301 (FIG. 3A) and sub-menus 302, 303 (FIGS. 3B-C) implemented with a scroller construct, as described above with respect to FIGS. 2A-D. For example, a user may touch “parameter settings” 310 in the main menu scroller 301 and be presented with a parameter menu stroller 302. The user may then touch “alarm limits” 320 in the parameter settings stroller 302 and be presented with the alarm limits scroller 303.

FIGS. 4A-C illustrate a spinner having one or more tiers, which open one at a time. Shown is a two-tiered spinner 400. Each spinner tier 410, 420 can display any specified number. The user applies touch to open one tier of the spinner at a time. The spinner elements include a label 430, buttons 440 and corresponding button text. A tier open state 401 (FIG. 4B), 402 (FIG. 4C) has two preceding and two trailing values on a spinner element, and a spinner closed state 403 (FIG. 4B) displaying the selected value. In the spinner open state, the user can use a vertical touch and move or flick to adjust the value. When open, a spinner tier overlays other user controls on the screen. To close the spinner the user can touch the center, highlighted value or another control on the screen.

FIGS. 5A-B illustrate a slider 500 that allows one touch value settings, such as for parameter limits as one example. FIG. 6 illustrates a slider-spinner 600 embodiment, which is a combination of a slider and spinner, each described separately above. A slider-spinner 600 advantageously allows both a quick and an accurate capability to set a value. In particular, the slider 601 allows a user to quickly get to a specific range and the spinner allows a fine adjustment of that range.

FIGS. 7A-D illustrate a scalable parameter display 700 and corresponding parameter well 710 advantageously providing a parameter monitor touchscreen with dynamic allocation of the parameter display area 700 so as to maximize screen capability and a caregiver's ability to automatically emphasize and distinguish parameters of greater importance from parameters of lesser importance. In particular, different monitor users care about different parameters. For example, a hemotologist might focus on blood-related parameters, such as SpHb, a noninvasive and continuous reading of total hemoglobin. Accordingly, the user has the ability to remove parameters of little or no interest from a main display area 700 and to place them in the parameter well 710. This is accomplished by a touch and hold gesture over a parameter to select the parameter, followed by a drag and drop gesture to remove the selected parameter from the main display area 700 into the well 710. The parameters remaining in the main display area 700 become bigger in size according to the number of remaining parameters. The removed parameters become smaller in size according to the number of parameters in the well 710. That is, the monitor dynamically adjusts parameter size according the available main display and well display areas. For example, FIG. 7A illustrates eight parameters in the main display 700 and one parameter (SpOC) in the well 710. FIG. 7B illustrates the relative size of six parameters in the main display 700, with three parameters in the well 710. FIG. 7C illustrates three parameters in the main display 700 dynamically increasing in size and six parameters in the well 710. FIG. 7D illustrates a single, very large SpO2 parameter advantageously solely displayed 700 so as to provide particular emphasis to that parameter and in a manner that can be seen across a room and readily noticed and monitored for change even by caregivers passing by at a distance. Sensors trigger parameters that are displayed so as not to hold space for non-active parameters.

A physiological monitor touchscreen interface has been disclosed in detail in connection with various embodiments. These embodiments are disclosed by way of examples only and are not to limit the scope of the claims herein. One of ordinary skill in art will appreciate many variations and modifications.

1.-20. (canceled)

21. A physiological monitor comprising:
a touchscreen display;
one or more physiological sensors configured to provide signals corresponding to a plurality of physiological parameters; and
one or more processors configured to:
present, on the touchscreen display, a menu interface including a menu item associated with alarm limits for at least one physiological parameter;
present, in response to selection of the menu item, an alarm setting interface including a slider operable to set an alarm limit value for the at least one physiological parameter and a value selector operable to refine the alarm limit value set by the slider;
display physiological parameters in a main display area and a secondary display area of the touchscreen display; and
dynamically adjust a display size of one or more displayed physiological parameters in response to movement of at least one physiological parameter between the main display area and the secondary display area.

22. The physiological monitor of claim 21, wherein the menu interface comprises: a first scroller configured to present a rotating set of menu items in a touchscreen display area; and
a second scroller configured to present a rotating set of thumbnails in a display well, wherein the thumbnails reference the menu items and rotate with the menu items.

23. The physiological monitor of claim 22, wherein the first scroller is configured to present a rotating set of second level menu items upon selection of the menu item.

24. The physiological monitor of claim 23, wherein the second level menu items include an alarm limits menu item.

25. The physiological monitor of claim 21, wherein the slider is configured to provide a gross alarm limit selection.

26. The physiological monitor of claim 25, wherein the value selector is configured to provide a finer alarm limit selection than the slider.

27. The physiological monitor of claim 26, wherein the value selector comprises a spinner.

28. The physiological monitor of claim 27, wherein the spinner comprises a first tier configured to define a first alarm limit and a second tier configured to define a second alarm limit.

29. The physiological monitor of claim 21, wherein the main display area is configured to display physiological parameter values in a full presentation format and the secondary display area is configured to display physiological parameter values in an abbreviated presentation format.

30. The physiological monitor of claim 29, wherein the full presentation format comprises a larger font than the abbreviated presentation format.

31. The physiological monitor of claim 21, wherein the one or more processors are configured to move a selected physiological parameter from the main display area to the secondary display area in response to a touch-and-hold gesture followed by a drag-and-drop gesture.

32. The physiological monitor of claim 21, wherein the one or more processors are configured to display physiological parameters responsive to sensor triggering so as not to hold space for non-active physiological parameters.

33. The physiological monitor of claim 21, wherein the one or more processors are configured to enlarge a selected physiological parameter such that the selected physiological parameter occupies substantially the main display area.

34. The physiological monitor of claim 21, wherein the main display area comprises a parameter area and the secondary display area comprises a parameter well area.

35. A method of operating a physiological monitor having a touchscreen display and one or more physiological sensors, the method comprising:
presenting, on the touchscreen display, a menu interface including a menu item associated with alarm limits for at least one physiological parameter;
receiving a gesture-based selection of the menu item;
presenting, in response to the selection, an alarm setting interface including a slider and a value selector;
receiving an input to the slider to set an alarm limit value for the at least one physiological parameter;
presenting the value selector to refine the alarm limit value set by the slider;
displaying physiological parameters in a main display area and a secondary display area of the touchscreen display; and
dynamically adjusting a display size of one or more displayed physiological parameters in response to movement of at least one physiological parameter between the main display area and the secondary display area.

36. The method of claim 35, wherein presenting the menu interface comprises presenting a virtually rotating menu scroller, and wherein receiving the gesture-based selection comprises receiving a flick on a first icon of the menu scroller so as to rotate the menu scroller.

37. The method of claim 36, further comprising receiving a touch on a second icon on the menu scroller so as to open a corresponding menu selection.

38. The method of claim 37, further comprising receiving a touch on a thumbnail within a thumbnail scroller synchronized with the menu scroller so as to open the corresponding menu selection.

39. The method of claim 35, wherein the value selector comprises a spinner having a first tier to define a first alarm limit and a second tier to define a second alarm limit.

40. The method of claim 35, further comprising displaying physiological parameters responsive to sensor triggering so as not to hold space for non-active physiological parameters.
